(19) United States
(12) Patent Application Publication
(10) Pub. No.: US 2022/0093246 A1
(43) Pub. Date: Mar. 24, 2022

(54) MITIGATING ACTIVITY DEFICIENCY

(71) Applicant: International Business Machines Corporation, Armonk, NY (US)

(72) Inventors: Venkata Vara Prasad Karri, Visakhapatnam (IN); Sarbajit K. Rakshit, Kolkata (IN); Akash U. Dhoot, Pune (IN); Shailendra Moyal, Pune (IN)

(21) Appl. No.: 17/029,386

(22) Filed: Sep. 23, 2020

(51) Int. Cl. G16H 40/67; G16H 20/30; G16H 10/60; H04L 29/08; G16Y 20/40; G16Y 10/75

(52) U.S. Cl. CPC G16H 40/67; G16H 20/30; G16H 10/60; G16Y 10/75; H04L 67/22; G16Y 20/40; H04L 67/306

(57) ABSTRACT

A computer-implemented method for mitigating activity deficiency comprising processors configured for receiving user profile data associated with a user profile, the user profile data comprising demographic data, past activity data, and health data. The method may include receiving device profile data comprising device schedule data corresponding to IoT devices; determining device capabilities based on the device profile data, wherein the device capabilities are configured to be performed by each of the IoT devices based on the device schedule data; receiving present activity data associated with the user profile; determining device state data corresponding to each of the IoT devices; determining actions to be performed associated with the IoT devices based on the user profile data, the device profile data, the present activity data and the device state data; and sending output data representing the actions to be performed to a user device associated with the user profile.

### BACKGROUND

The present invention relates generally to the field of mitigating activity deficiency, and more particularly to recommending user activity to mitigate user activity deficiency.

Internet of Things (IoT) refers to the concept of extending internet connectivity beyond conventional computing platforms such as personal computers and mobile devices, and into any range of traditionally non-internet-enabled physical devices and everyday objects. Embedded with electronics, internet connectivity, and other forms of hardware (such as sensors), these devices and objects can communicate and interact with others over the Internet, and the devices and objects can be remotely monitored and controlled.

The definition of IoT has evolved due to convergence of multiple technologies, real-time analytics, machine learning, commodity sensors, and embedded systems. Traditional fields of embedded systems, wireless sensor networks, control systems, automation (including home and building automation), and others all contribute to enabling the IoT. In the consumer market, IoT technology is most synonymous with products pertaining to the concept of the “smart home,” covering devices and appliances (such as lighting fixtures, thermostats, home security systems and cameras, and other home appliances) that support one or more common ecosystems, and can be controlled via devices associated with that ecosystem, such as smartphones and smart speakers.

### SUMMARY

Aspects of an embodiment of the present invention disclose a method, computer program product, and computer system for mitigating activity deficiencies of a user. One embodiment of the invention may include receiving user profile data associated with a user profile, the user profile data comprising demographic data, past activity data, and health data; receiving device profile data comprising device schedule data corresponding to one or more devices; determining one or more device capabilities based on the device profile data, wherein the one or more device capabilities are configured to be performed by each of the one or more devices based on the device schedule data; receiving present activity data associated with the user profile; determining device state data corresponding to each of the one or more devices; determining one or more actions to be performed associated with at least one of the one or more devices based on the user profile data, the device profile data, the present activity data, and the device state data; and sending output data representing the one or more actions to be performed to a user device associated with the user profile.

The one or more devices may include an IoT device having one or more sensors configured to detect user activity comprising the past activity data. The one or more processors may further be configured for receiving the present activity data from the user device, wherein the present activity data is based on user activity received via one or more sensors of the user device. The device state data may correspond to a condition of the one or more devices, wherein the condition indicates a state or status of the one or more devices. The one or more actions may include an instruction to perform the one or more actions manually. The one or more device capabilities may be automatically performed by the one or more devices according to a device schedule represented in the device schedule data. The user profile data may further include user schedule data corresponding to periods of time having either an entry or no entry for each period of time.

### BRIEF DESCRIPTION OF THE DRAWINGS

FIG. 1 depicts a block diagram of a distributed data processing environment for mitigating activity deficiency, in accordance with an embodiment of the present invention.

FIG. 2 depicts a block diagram of a distributed data processing environment for mitigating activity deficiency, in accordance with another embodiment of the present invention.

FIG. 3 depicts a flowchart of a method for mitigating activity deficiency, in accordance with an embodiment of the present invention.

FIG. 4 depicts a block diagram of a computing device of distributed data processing environment, in accordance with an embodiment of the present invention.

### DETAILED DESCRIPTION

In a smart home environment, IoT devices may be programmed to automatically perform activities within the home in lieu of a resident performing the activities. However, during times where residents are confined to their homes for extended period of time, residents may suffer from a lack of outdoor activities. A solution is needed to mitigate the negative effects of reduced physical activity, which could lead to chronic health problems.

To mitigate activity deficiency, a solution is provided that gathers and processes data corresponding to user devices and IoT devices to determine what actions can be performed within a controlled environment. For example, if various members of a home environment are identified and data corresponding to their activities within and around the home is gathered and processed, recommendations can be suggested to them on what activities can be performed to make up for a determined lack of exercise or movement activity. Such recommendations may reduce the chance of health-related problems that may be predicted to occur in the future due to the lack of exercise or movement activity.

Aspects of the invention described herein provide the ability to suggest actions to be performed to mitigate activity deficiency. Certain aspects of this invention's embodiments may include receiving data corresponding to user device information, user information, and IoT device information, and determining a recommended action to perform based on the received data. The recommended action may be determined based on a certain user's health profile data and what types of activities should be done within a home environment to maintain the home environment.

Embodiments described herein include methods to identify users within a particular home environment, wherein each user may provide information or be provided with information to create a user profile. Information used to create a user profile may include demographic data, biometric data, health data, past activity data, present activity data, user schedule data, or other types of data corresponding to the user and/or the associated user device. Once each user profile is established, a user's fitness level may be assessed based on user profile data and particular activities may be suggested to the user via the respective user device. The activities may include exercise or chores to be performed within the home environment. Artificial intelligence methods may be used to receive all the relevant data and generate output data corresponding to which user should perform which activity or action. Data corresponding to IoT devices within the home environment may also be gathered and processed to determine the state or status of the IoT devices and which actions the IoT devices are configured to perform. The type of activity or action and the number of minimum calories to burn or that can be burned may be determined based on user profile data and IoT device data. For example, if user profile data indicates that a first user would benefit from burning 500 calories during a period of time, and IoT device data indicates that one or more IoT devices will automatically perform activities totaling 500 calories according to device schedule data in the IoT device data, then one or more processors may be configured to process the user profile data and the IoT device data to generate output data including instructions to the user device associated with the user profile to manually perform the identified activities. Further, user profile data may be used to identify potential health problems specific to the user, wherein manually performing the identified activities may decrease the chance of the user suffering from or treating the symptoms of the identified health problems.

An embodiment may include IoT devices gathering past activity data over a period of time. Past activity data may be gathered by IoT devices, user devices, or other devices or sensors configured to detect and gather activity within the home environment. Past activity data may include data gathered while the user was cleaning floors, moving around a room, cooking, preparation for cooking, arranging home items or furniture, etc. Past activity data may also include data gathered while the user was performing chores inside or outside of the home environment.

Furthermore, embodiments described herein may track user activity needs, identify how the lack of user activity may create health problems, and assign appropriate physical activities to users within the home environment based on that particular user's needs. Described embodiments may be implemented within a smart home environment, wherein one or more processors within the smart home environment may be configured to determine if an automated IoT device or other connected device can be stopped from performing its automated functions so that the stopped function may be assigned to a user via a user device, wherein performing the function may improve the health of the user. Certain embodiments may also include one or more processors configured for scheduling activities to be performed by users within the smart home environment by gathering device schedule data and user profile data to determine a time when the activity should be performed during a time wherein the user has an opening in their schedule. For example, a first user may receive an instruction on an associated user device to perform a first action and a second user may receive another instruction on another associated user device to perform a second action, wherein each instruction may include a specific time and/or frequency upon which the actions should be performed.

Aspects of an example embodiment of the present invention disclose a computer-implemented method, computer program product, and computer system for mitigating activity deficiencies of a user. One embodiment of the invention may include receiving user profile data associated with a user profile, the user profile data including demographic data, past activity data, and health data; receiving device profile data including device schedule data corresponding to one or more devices; determining one or more device capabilities based on the device profile data, wherein the one or more device capabilities are configured to be performed by each of the one or more devices based on the device schedule data; receiving present activity data associated with the user profile; determining device state data corresponding to each of the one or more devices; processing the user profile data, the device profile data, the present activity data, and the device state data; determining one or more actions to be performed associated with at least one of the one or more devices based on the user profile data, the device profile data, the present activity data, and the device state data; and sending output data representing the one or more actions to be performed to a user device associated with the user profile.

The one or more devices may include an IoT device having one or more sensors configured to detect user activity, wherein the user activity may include past activity data. The one or more processors may further be configured for receiving the present activity data from the user device, wherein the present activity data is based on user activity received via one or more sensors of the user device. The device state data may correspond to a condition of one of the one or more devices. The one or more actions may include an instruction to perform the one or more actions manually. The one or more device capabilities may be automatically performed by the one or more devices according to a device schedule represented in the device schedule data. The user profile data may further include user schedule data corresponding to periods of time having either an entry or no entry for each period of time.

The present invention will now be described in detail with reference to the Figures.

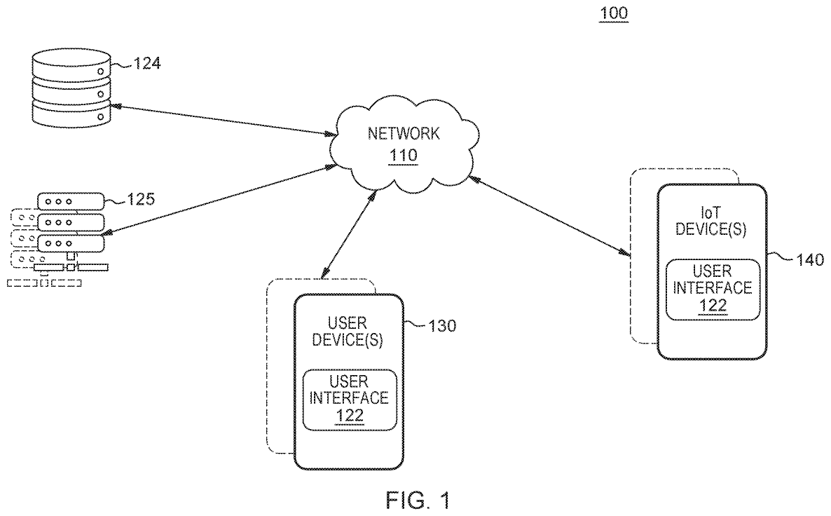

FIG. 1 depicts a block diagram of a distributed data processing environment 100 for mitigating activity deficiency, in accordance with an embodiment of the present invention.

FIG. 1 provides only an illustration of one embodiment of the present invention and does not imply any limitations with regard to the environments in which different embodiments may be implemented. As shown in FIG. 1, the distributed data processing environment 100 for mitigating activity deficiency includes network 110 configured to facilitate communication between database 124, server 125, user device(s) 130 and IoT device(s) 140. In an example embodiment, one or more processors may be configured for receiving user profile data, device profile data, and present activity data via network 110. Further, one or more processors may be configured for sending output data to user device(s) 130 via network 110.

Network 110 operates as a computing network that can be, for example, a local area network (LAN), a wide area network (WAN), or a combination of the two, and can include wired, wireless, or fiber optic connections. In general, network 110 can be any combination of connections and protocols that will support communications between user device(s) 130 and IoT device(s) 140. It is further understood that in some embodiments network 110 is optional and the distributed data processing environment 100 for mitigating activity deficiency can operate as a stand-alone system, where in other embodiments, network 110 may be configured to enable user device(s) 130 and/or IoT device(s) 140 to share a joint database using network 110.

User interface 122 operates as a local user interface on user device(s) 130 through which one or more users of user device(s) 130 interact with user device(s) 130. User interface 122 may also operate as a local user interface on IoT device(s) 140 through which one or more users of IoT device(s) 130 interact with IoT device(s) 140. In some embodiments, user interface 122 is a local app interface of a program (e.g., software configured to execute the steps of the invention described herein) on user device(s) 130 or IoT device(s) 140. In some embodiments, user interface 122 is a graphical user interface (GUI), a web user interface (WUI), and/or a voice user interface (VUI) that can display (i.e., visually), present (i.e., audibly), and/or enable a user to enter or receive information (i.e., graphics, text, and/or sound) for or from the program via network 110. In an embodiment, user interface 122 enables a user to send and receive data (i.e., to and from the program via network 110, respectively). In an embodiment, user interface 122 enables a user to opt-in to the program, input user related data, and receive alerts to complete a task or activity.

Database 124 may operate as a repository for data associated with server 125, user device(s) 130, IoT device(s) 140, and other data transmitted within network 110. A database is an organized collection of data. For example, user profile data may include data associated with user device(s) 130. Further, device profile data may include data associated with IoT device(s) 140. Database 124 can be implemented with any type of storage device capable of storing data and configuration files that can be accessed and utilized by either of user device(s) 130 or IoT device(s) 140, such as a database server, a hard disk drive, or a flash memory. In an embodiment, database 124 may be accessed by user device(s) 130 or IoT device(s) 140 to store data associated with user device(s) 130 or IoT device(s) 140. In another embodiment, database 124 may be accessed by user device(s) 130 or IoT device(s) 140 to access data as described herein. In an embodiment, database 124 may reside independent of network 110. In another embodiment, database 124 may reside elsewhere within distributed data processing environment 100 provided database 124 has access to network 110.

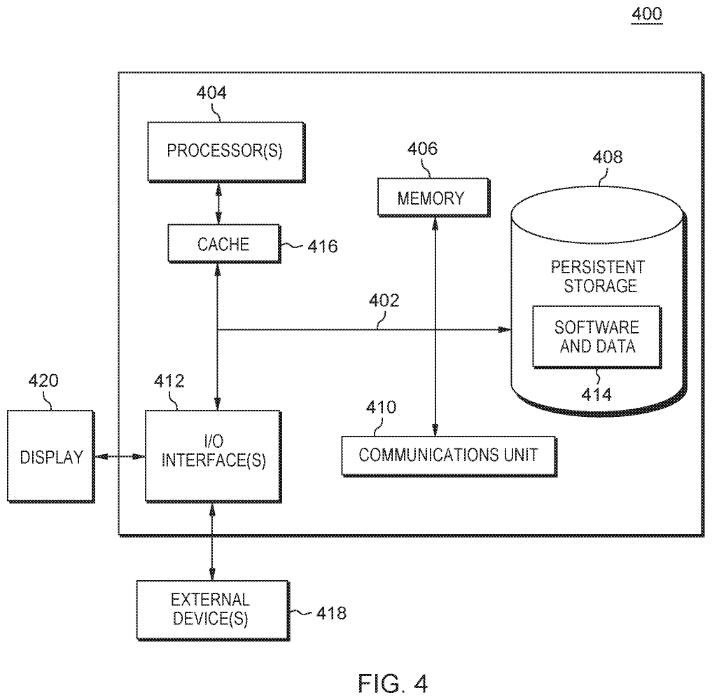

In the depicted embodiment, server(s) 125 may contain a program (e.g., software configured to execute the steps of the invention described herein) and database 124. In some embodiments, server(s) 125 can be a standalone computing device(s), a management server(s), a web server(s), a mobile computing device(s), or any other electronic device(s) or computing system(s) capable of receiving, sending, and processing data. In some embodiments, server 125 may be a laptop computer, tablet computer, netbook computer, personal computer (PC), a desktop computer, a smart phone, or any programmable electronic device capable of communicating with devices 120, 130 via network 110. In other embodiments, server(s) 125 represents a server computing system utilizing multiple computers as a server system, such as a cloud computing environment. In yet other embodiments, server(s) 125 represents a computing system utilizing clustered computers and components (e.g., database server computers, application server computers, etc.) that act as a single pool of seamless resources when accessed within distributed data processing environment 100. Server(s) 125 may include components as described in further detail in FIG. 4.

User device(s) 130 may be an electronic device configured for accompaniment with a user. User device(s) 130 may be a personal electronic device such as a mobile communications device, smart phone, tablet, personal digital assistant, smart wearable device, personal laptop computer, desktop computer, or any other electronic device configured for user interaction and gathering user information to generate a user profile. User device(s) 130 may include components as described in further detail in FIG. 4.

IoT device(s) 140 may be an electronic device configured to be a component within a smart home automation system including lighting systems, heating and air conditioning systems, media, and security systems. The electronic device may include a wireless sensor, software, actuators, and computer devices. IoT device(s) 140 may be embedded in mobile devices, industrial equipment, environmental sensors, medical devices, and others. IoT device(s) 140 may be controlled from a remotely controlled system via network 110 or locally controlled system via a local network, or a combination of both. Further, IoT device(s) 140 may be configured to be controlled via a software application installed and executed by IoT device(s) 140. IoT device(s) 140, when connected to a network, may convey usage data and other types of data corresponding to the device itself, or other devices connected via network 110, wherein the data may provide insights that are useful within the scope of the designed application. configured with a processor, memory, and peripherals (not shown) to receive and process data.

For user device(s) 130, a device profile includes, but is not limited to, a user device identifier (ID), a device type (e.g., a smart watch, a smart phone), data usage patterns for user device(s) 130, and data usage models for user device(s) 130. Data usage patterns may include data type, data use frequency, and user device data use history. A device profile may be created for each user device 130 in network 110. User device(s) 130 may consider data usage patterns and data usage models in a device profile when determining whether to execute a data usage request by user device(s) 130.

For IoT device(s) 140, a device profile includes, but is not limited to, an IoT device identifier (ID), an IoT device type (e.g., a smart lock, smart dishwasher), data usage patterns for IoT device(s) 140, and data usage models for IoT device(s) 140. Data usage patterns may include data type, data use frequency, and IoT device data use history. A device profile may be created for each IoT device(s) 140 in network 110. IoT device(s) 140 may consider data usage patterns and data usage models in a device profile when determining whether to execute a data usage request by IoT device(s) 140.

User device(s) 130 and/or IoT device(s) 140 may operate as physical devices and/or everyday objects that are embedded with electronics, Internet connectivity, and other forms of hardware (e.g., sensors). In general, IoT device(s) 140 can communicate and interact with other IoT device(s) 140 over the Internet or a local network while being remotely monitored and controlled. Types of IoT device(s) 140 include, but are not limited to, smart locks, garage doors, refrigerators, freezers, ovens, mobile devices, smart watches, air conditioning (A/C) units, washer/dryer units, smart TVs, virtual assistance devices, and any other smart home devices.

In an embodiment, a user may be permitted to opt-in and/or agree to a terms and service agreement upon setting up IoT devices with network 110. The terms and service agreement may document the purpose of the information and data sharing between user device(s) 130 or IoT device(s) 140 and provide access to IoT devices on the network that have been designated for participation in network 110. The user agreement may include all mentioned passing devices that would allow control(s), trigger(s), or action(s) to be executed based on the user's original request. For networks with multiple users and multiple IoT devices, the system may extend the usage agreement to a defined or dynamic group, upon a new user joining said group.

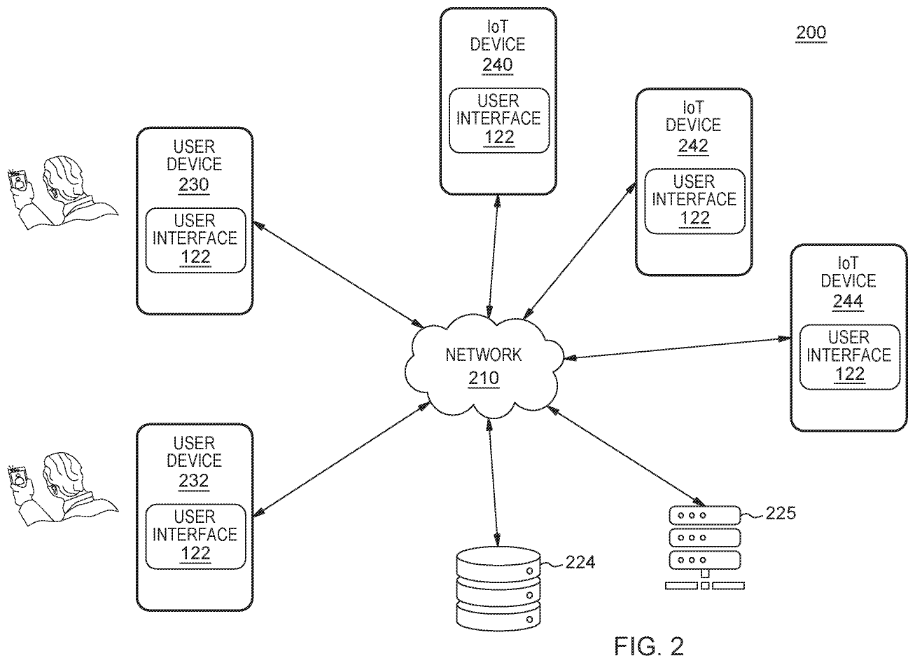

FIG. 2 depicts a block diagram of a distributed data processing environment 200 for mitigating activity deficiency, in accordance with another embodiment of the present invention.

In an embodiment, distributed data processing environment 200 may include network 210 configured for facilitating communication between one or more user devices (e.g., user device 230, user device 232) and one or more IoT devices (e.g., IoT device 240, IoT device 242, IoT device 244). User device 230 and user device 232 may be configured to operate similar to how user device(s) 130 are described to operate in FIG. 1. IoT device 240, IoT device 242, and IoT device 244 may be configured to operate similar to how IoT device(s) 140 are described to operate in FIG. 1. Distributed data processing environment 200 may further include database 224 configured for storing data transmitted via network 210 and server 225 configured for processing data transmitted via network 210. For example, IoT devices (e.g., IoT device 240, IoT device 242, and IoT device 244) of a given household may be placed throughout and connected to network 110 (e.g., Wi-Fi network). IoT devices (e.g., IoT device 240, IoT device 242, and IoT device 244) connected to network 110 may include local communication ports that may be in an open state to accept text data and treat the text data as text input to a logic engine. The connection to the Wi-Fi network may setup similarly to how a user may setup a smart plug or smart lightbulb, as known to a person of ordinary skill in the art.

In an embodiment, one or more processors may be configured to receive historical data from one or more of user device 230, user device 232, IoT device 240, IoT device 242, and IoT device 244 to determine a pattern of activities performed by a user associated with one of user device 230 or user device 232 and a pattern of actions or functions performed by IoT device 240, IoT device 242, and/or IoT device 244. As a pattern of activities and/or functions may be identified over a period of time, the one or more processors may be configured to determine a time duration in which an activity should be performed by a user associated with one of user device 230 or user device 232. The time duration may be determined based on the historical data corresponding to the amount of time it took for the activity to be performed by a particular user and other factors.

Further, the one or more processors may be configured to associate a period of time in which to perform the activity with at least one unit of time available in a device schedule for one of IoT device 240, IoT device 242, or IoT device 244. In other words, the identified time duration to perform the activity may be assigned to a unit of time of a device schedule for one of IoT device 240, IoT device 242, or IoT device 244. Thus, once associated with the device schedule, the identified activity may be shown as a pending activity to be performed either automatically by one of IoT device 240, IoT device 242, or IoT device 244 or assigned to be performed by a user associated with one of user device 230 or user device 232.

In an embodiment, the one or more processors may be configured to determine a number of calories to be burned by a user associated with one of user device 230 or user device 232. For example, user profile data for a user associated with user device 230 may be processed to determine that the user should burn 1000 calories in a day. The number of calories may be determined based on historical data and user profile data received from one or more of user device 230 and user device 232.

In an embodiment, the one or more processors may be configured to determine one or more user skills to be performed by a user associated with one of user device 230 or user device 232. For example, user profile data for a user associated with user device 230 may be processed to determine that the user is skilled with the ability to perform one or more activities. The one or more activities may be determined based on historical data and user profile data received from one or more of user device 230 and user device 232.

In an embodiment, the one or more processors may be configured to determine a number of available units of time for a user associated with one of user device 230 or user device 232 to perform one or more activities. For example, user profile data for a user associated with user device 230 may be processed to determine that the user should burn 1000 calories in a day. The number of calories may be determined based on historical data and user profile data received from one or more of user device 230 and user device 232.

Once the number of available units of time is determined for the user, the one or more processors may be configured for determining which identified activities or functions for one or more IoT devices may be assigned to the user to be performed during the available units of time. For example, if the one or more processors determine that IoT device 240 has a pending activity to be performed in 1 unit of time, and the one or more processors determine that a user associated with user device 230 has a skill compatible with the pending activity and has 1 unit of time available in the user schedule, then the one or more processors may be configured to send an instruction to user device 230 to assign the pending activity to the user. The instruction may include information about the pending activity including the number of calories that can be burned by the user performing the activity, the number of units of time it will take user to perform the activity, and other health related benefits of user performing the activity and health related risks of not performing the activity.

In an embodiment, the instruction may also include manual instructions on how to perform the activity to assist the user in performing the activity in such a way to gain the most benefit from performing the activity. The manual instructions may be in the form of step-by-step instructions displayed as text on a user interface display. The manual instructions may also be in the form of Augmented Reality (“AR”) gamification demonstrating how to perform the activity. For example, the one or more processors may be configured to play music along with the AR gamification, in addition to the demonstration being performed by an animated character or avatar configured to guide the user to get the user involved in performing the activity. In another embodiment, the AR gamification may also be configured to perform verbal communications to communicate with the user to assist the user in performing the activity. The AR gamification may be generated within a glass medium to produce images corresponding to the animated character or avatar, as described herein.

In another embodiment, if the user is determined to not have the number of units of time available to perform the activity, then the one or more processors may be configured to send an instruction to the IoT device identified as having the device capability to perform the activity instead. In other words, if the user is not available to perform the activity within the period of time that the activity should be performed, then the IoT device may be configured to automatically perform the corresponding activity according to the device schedule.

In another embodiment, if the user is determined to only have a portion of the number of units of time available to perform the activity, then the one or more processors may be configured to send an instruction to the IoT device identified as having the device capability to perform a portion of the activity and send an instruction to user device 230 associated with the user to perform the remaining portion of the activity within the portion of the number of units of time available. In other words, the user and the IoT device may share the responsibility of performing the activity in the event that the user's available time permits the shared activity to be performed.

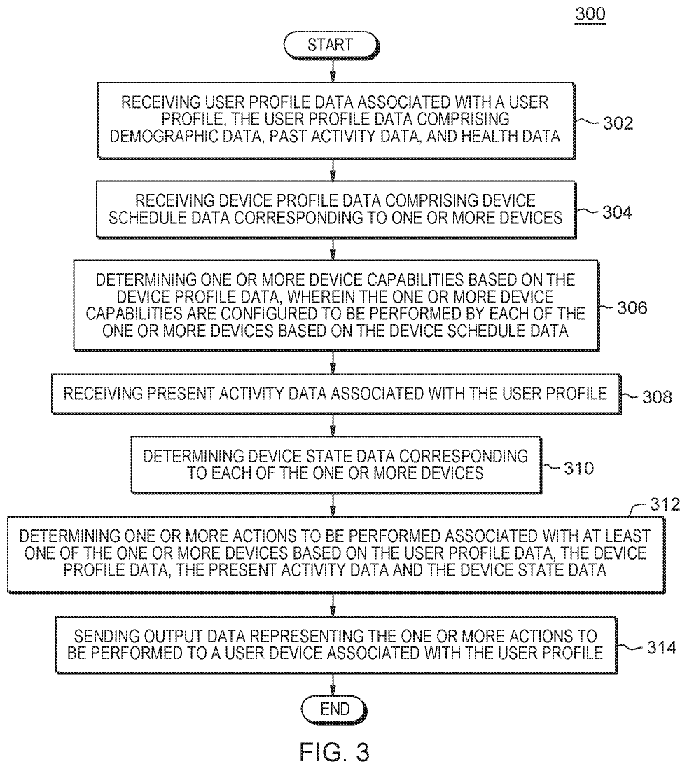

FIG. 3 depicts a flowchart of a computer-implemented method 300 for mitigating activity deficiency, in accordance with an embodiment of the present invention.

In an embodiment, computer-implemented method 300 may include one or more processors configured for receiving 302 user profile data associated with a user profile, wherein the user profile data may include demographic data, past activity data, and health data. For example, user device 130 may include a user interface configured for receiving user inputs corresponding to data associated with a user. The user inputs may be stored as user profile data and associated with a user profile linked with user device 130. User profile data may include biometric data, biographical data, health data, past activity data, and other data attributable to a user of user device. In another embodiment, user profile data may be attributable to another user who is not necessarily a user of user device but may be a user known to the user of user device 130. User profile data may be gathered via sensors of user device 130, entered via a user interface of user device 130, or received from another source or database via a communications link to user device 130.

User profile data may also include a user schedule corresponding to units of time for each day of the week including either an event or no event. For example, a user schedule may include 24 equal units of time representing each hour of the day, wherein each unit of time may be partitioned into sub-units of time that are less than the unit of time. Each unit of time may include an event scheduled for an action or an activity, whereas unscheduled units of time may be unoccupied as a no event entry. A user schedule may include an entry for a unit of time for one or more actions or activities, wherein the entry may be made by the user or suggested by a third party and accepted by the user. The user schedule may include an entry for multiple units of time designated for work, sleep, or other activities. Other activities may include activities within the home environment or leisure activities including exercise or extracurricular activities.

Further, computer-implemented method 300 may include one or more processors configured for receiving 304 device profile data comprising device schedule data corresponding to one or more devices, wherein the one or more devices may include IoT device 140. Device profile data may include data corresponding to features and characteristics of IoT device 140. For example, if IoT device 140 is a smart dishwasher, then device profile data for the smart dishwasher may include feature data and specification data. Feature data may include data corresponding to characteristics (e.g., model number, system identifiers, technology identifiers, etc.) and unique to the smart dishwasher. Feature data may also include capabilities or functionalities of IoT device 140. Capabilities or functionalities may correspond to specific actions or functions that IoT device 140 is configured to perform. Specification data may include type, style, physical dimensions, energy star ratings, connectivity options, and other manufacturer specifications.

Device profile data may also include device schedule data that includes 24 equal units of time representing each hour of the day, wherein each unit of time may be partitioned into sub-units of time that are less than the unit of time. Each unit of time may include an event scheduled for an action or a function, whereas unscheduled units of time may be unoccupied as a no event entry. A device schedule may include an entry for a unit of time for one or more actions or functions, wherein the entry may be made by receiving data configured to create an entry via, e.g., network 110 or directly via a user interface connected to IoT device 140.

Further, computer-implemented method 300 may include one or more processors configured for determining 306 one or more device capabilities based on the device profile data, wherein the one or more device capabilities may be configured to be performed by each of the one or more devices based on the device schedule data. Device capabilities may be determined by one or more processors based on device profile data, wherein the device profile data may be processed, and one or more device capabilities are identified. For example, IoT device 140 may be configured to receive and store device profile data corresponding to features and characteristics of IoT device 140, which when processed by one or more processors may identify one or more device capabilities of IoT device 140.

Once identified, the IoT device may be configured to execute or perform the one or more device capabilities when a command to execute or perform is received. For example, if IoT device 140 is a smart dish washer and the one or more capabilities identified based on the device profile data of the smart dishwasher is a wash cycle, then IoT device 140 will perform the wash cycle once it receives the command to do so.

Further, computer-implemented method 300 may include one or more processors configured for receiving 308 present activity data associated with the user profile. Present activity data may include data corresponding to activity or movement currently being performed by the user associated with the user profile. For example, if a user is detected as walking or cleaning the floor, user device 130 and/or IoT device 140 may be configured to detect the user walking or cleaning the floor as present user activity and identify each activity as such.

Further, computer-implemented method 300 may include one or more processors configured for determining 310 device state data corresponding to each of the one or more devices. Device state data may include data corresponding to the state or condition of an IoT device at a particular time. Device states for the smart dishwasher may include status for occupancy, content condition, availability, and other manufacture provided statuses or conditions. For example, IoT device 140 may be a smart dishwasher configured to determine that its occupancy is either empty or occupied, its content condition may be either clean or dirty, and its availability may be in use or idle. Other types of IoT devices may include other device states based on the IoT device manufacturer's specifications or settings.

Further, computer-implemented method 300 may include one or more processors configured for processing the user profile data, the device profile data, the present activity data and the device state data. In an embodiment, one or more processors may be configured to initialize a machine learning model to receive a data set including at least one of the user profile data, the device profile data, the present activity data and the device state data, generate, for each data string in the data set, multiple feature vectors corresponding, respectively, to each data string in the data set.

Once the multiple feature vectors are generated, the one or more processors may be configured to group the feature vectors to form a group of feature vectors such that each feature vector of the group of feature vectors corresponds to one type of data from the data set. The group of feature vectors may be concatenated to form an input feature vector to be provided to a machine learning model as training data to train the machine learning model.

Further, computer-implemented method 300 may include one or more processors configured for determining 312 one or more actions to be performed associated with at least one of the one or more devices based on the user profile data, the device profile data, the present activity data, and the device state data. For example, the one or more devices may include IoT device 140. In an embodiment, the one or more processors may be configured to initialize a machine learning model to determine which user device should receive an instruction including an action to be performed by a user associated with the user device, wherein the action may be an action that is one of the one or more capabilities of the IoT device.

In an embodiment, machine learning model may also be configured to learn user activity (e.g., basic routines) at least by mining data received in the data sets described herein. Machine learning model may also be configured to establish a user health baseline based on data corresponding to user characteristics (e.g., patient health data, patient biometric data, and patient biographical data), wherein user characteristics may include age, weight, previous medical conditions, medical history, etc. Machine learning model may be configured to detect abnormalities in sensor readings and determine what activity should be recommended to reduce the potential effects of the detected abnormalities. Machine learning model may also be configured to perform predictive modeling by forecasting deviations in biometric readings and to predict future conditions and recommend activity to reduce the probability of those future conditions from occurring.

Further, computer-implemented method 300 may include one or more processors configured for sending 314 output data representing the one or more actions to be performed to a user device associated with the user profile. For example, the one or more processors may be configured for initializing a machine learning model to process the data set and output data corresponding to a recommendation or suggestion of a user associated with user device 130 to perform an activity corresponding to a function that is automatically performed by IoT device 140. The recommendation may include a message shown on a display providing information corresponding to the activity, including instructions on how to perform the activity and other information about benefits (e.g., calorie value, health benefits, manual instructions) of performing the activity. The recommendation may also include a time period during which the activity should be performed, which may be determined based on schedule data included in the user profile data and the device schedule data.

In another embodiment, the one or more devices may include an IoT device including one or more sensors configured to detect user activity comprising the past activity data.

In another embodiment, computer-implemented method 300 may include one or more processors configured for receiving the present activity data from the user device, wherein the present activity data may be based on user activity received via one or more sensors of the user device.

The device state data may correspond to at least a condition of at least one of the one or more devices, wherein the condition may indicate a state of the one or more devices. A state may include whether the one or more devices is on, off, occupied, empty, in use, not in use, clean, dirty, or any other condition indicative of the one or more devices.

The one or more actions may include an instruction to perform the one or more actions manually. For example, if the one or more actions is to clean the floor, the instruction sent to the user device may display or communicate a recommendation to clean the floor with a mop and/or a broom or manual floor cleaning apparatus.

The one or more device capabilities may be automatically performed by the one or more devices according to a device schedule represented in the device schedule data.

The user profile data may further include user schedule data corresponding to periods of time having either an entry or no entry for each period of time.

In an embodiment, one or more processors may be configured to execute a mobility tracking system configured to track user movement throughout a home environment. For example, the mobility tracking system may include one or more sensors embedded within IoT devices (e.g. IoT device 140, IoT device 240, IoT device 242, IoT device 244) affixed to common household items or appliances configured to detect user movement throughout the home environment or user use of the common household items or appliances. The detected user movement may be transmitted as present activity data to, e.g., network 110 to be attributed to the corresponding user profile associated with, e.g., user device 130.

FIG. 4 depicts a block diagram of computer 400 suitable for server 125, user device(s) 130, and/or IoT device(s) 140, in accordance with an illustrative embodiment of the present invention. It should be appreciated that FIG. 4 provides only an illustration of one implementation and does not imply any limitations with regard to the environments in which different embodiments may be implemented. Many modifications to the depicted environment may be made.

Computer 400 includes communications fabric 402, which provides communications between cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Software and data 414 may be stored in persistent storage 408 and in memory 406 for execution and/or access by one or more of the respective computer processors 404 via cache 416. In an embodiment, persistent storage 408 includes a magnetic hard disk drive. Alternatively, or in addition to a magnetic hard disk drive, persistent storage 408 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. Software and data 414 may be downloaded to persistent storage 408 through communications unit 410.

I/O interface(s) 412 allows for input and output of data with other devices that may be connected to server 125, user device(s) 130, and/or IoT device(s) 140. For example, I/O interface 412 may provide a connection to external devices 418 such as a keyboard, keypad, a touch screen, and/or some other suitable input device. External devices 418 can also include portable computer readable storage media such as, for example, thumb drives, portable optical or magnetic disks, and memory cards. Software and data 414 used to practice embodiments of the present invention can be stored on such portable computer readable storage media and can be loaded onto persistent storage 408 via I/O interface(s) 412. I/O interface(s) 412 also connect to a display 420.

Display 420 provides a mechanism to display data to a user and may be, for example, a computer monitor.

The present invention may contain various accessible data sources, such as database 124, that may include personal data, content, or information the user wishes not to be processed. Personal data includes personally identifying information or sensitive personal information as well as user information, such as tracking or geolocation information. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Software and data 414 may enable the authorized and secure processing of personal data. Software and data 414 may be configured to provide informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Software and data 414 may provide information regarding personal data and the nature (e.g., type, scope, purpose, duration, etc.) of the processing. Software and data 414 provide the user with copies of stored personal data. Software and data 414 allow the correction or completion of incorrect or incomplete personal data. Software and data 414 allow the immediate deletion of personal data.

The present invention may be a system, a computer-implemented method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of computer-implemented methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for mitigating activity deficiency, the method comprising:
receiving, by one or more processors, user profile data associated with a user profile, the user profile data comprising demographic data, past activity data, and health data;
receiving, by the one or more processors, device profile data comprising device schedule data corresponding to one or more devices;
determining, by the one or more processors, one or more device capabilities based on the device profile data, wherein the one or more device capabilities are configured to be performed by each of the one or more devices based on the device schedule data;
receiving, by the one or more processors, present activity data associated with the user profile;
determining, by the one or more processors, device state data corresponding to each of the one or more devices;
determining, by the one or more processors, one or more actions to be performed associated with at least one of the one or more devices based on the user profile data, the device profile data, the present activity data, and the device state data; and
sending, by the one or more processors, output data representing the one or more actions to be performed to a user device associated with the user profile.

2. The computer-implemented method of claim 1, wherein the one or more devices comprise an IoT device including one or more sensors configured to detect user activity comprising the past activity data.

3. The computer-implemented method of claim 1, wherein the present activity data is based on user activity received via one or more sensors of the user device.

4. The computer-implemented method of claim 1, wherein the device state data corresponds to a condition of at least one of the one or more devices.

5. The computer-implemented method of claim 1, wherein the one or more actions comprise an instruction to perform the one or more actions manually.

6. The computer-implemented method of claim 1, wherein the one or more device capabilities are automatically performed by the one or more devices according to a device schedule represented in the device schedule data.

7. The computer-implemented method of claim 1, wherein the user profile data further comprises user schedule data corresponding to periods of time having either an entry or no entry for each period of time.

8. A computer program product for mitigating activity deficiency, the computer program product comprising:
one or more computer readable storage media and program instructions stored on the one or more computer readable storage media, the program instructions comprising:
program instructions to receive user profile data associated with a user profile, the user profile data comprising demographic data, past activity data, and health data;
program instructions to receive device profile data comprising device schedule data corresponding to one or more devices;
program instructions to determine one or more device capabilities based on the device profile data, wherein the one or more device capabilities are configured to be performed by each of the one or more devices based on the device schedule data;
program instructions to receive present activity data associated with the user profile;
program instructions to determine device state data corresponding to each of the one or more devices;
program instructions to determine one or more actions to be performed associated with at least one of the one or more devices based on the user profile data, the device profile data, the present activity data and the device state data; and
program instructions to send output data representing the one or more actions to be performed to a user device associated with the user profile.

9. The computer program product of claim 8, wherein the one or more devices comprise an IoT device including one or more sensors configured to detect user activity comprising the past activity data.

10. The computer program product of claim 8, further comprising program instructions to receive the present activity data from the user device, wherein the present activity data is based on user activity received via one or more sensors of the user device.

11. The computer program product of claim 8, wherein the device state data corresponds to a condition of at least one of the one or more devices.

12. The computer program product of claim 8, wherein the one or more actions comprise an instruction to perform the one or more actions manually.

13. The computer program product of claim 8, wherein the one or more device capabilities are automatically performed by the one or more devices according to a device schedule represented in the device schedule data.

14. The computer program product of claim 8, wherein the user profile data further comprises user schedule data corresponding to periods of time having either an entry or no entry for each period of time.

15. A computer system for mitigating activity deficiency, the computer system comprising:
one or more computer processors;
one or more computer readable storage media;
program instructions stored on the one or more computer readable storage media for execution by at least one of the one or more processors, the program instructions comprising:
program instructions to receive user profile data associated with a user profile, the user profile data comprising demographic data, past activity data, and health data;
program instructions to receive device profile data comprising device schedule data corresponding to one or more devices;
program instructions to determine one or more device capabilities based on the device profile data, wherein the one or more device capabilities are configured to be performed by each of the one or more devices based on the device schedule data;
program instructions to receive present activity data associated with the user profile;
program instructions to determine device state data corresponding to each of the one or more devices;
program instructions to determine one or more actions to be performed associated with at least one of the one or more devices based on the user profile data, the device profile data, the present activity data and the device state data; and
program instructions to send output data representing the one or more actions to be performed to a user device associated with the user profile.

16. The computer system of claim 15, wherein the one or more devices comprise an IoT device including one or more sensors configured to detect user activity comprising the past activity data.

17. The computer system of claim 15, further comprising program instructions to receive the present activity data from the user device, wherein the present activity data is based on user activity received via one or more sensors of the user device.

18. The computer system of claim 15, wherein the device state data corresponds to at least to a condition of at least one of the one or more devices and the user profile data further comprise user schedule data corresponding to periods of time having either an entry or no entry for each period of time.

19. The computer system of claim 15, wherein the one or more actions comprise an instruction to perform the one or more actions manually.

20. The computer system of claim 15, wherein the one or more device capabilities are automatically performed by the one or more devices according to a device schedule represented in the device schedule data.
